(19) United States
(12) Patent Application Publication
(10) Pub. No.: US 2024/0280841 A1
(43) Pub. Date: Aug. 22, 2024

(54) Intra-Ocular Lens Camera

(71) Applicant: Ceyeber Corp., Newport Beach, CA (US)

(72) Inventor: Robert Edward Grant, Laguna Beach, CA (US)

(21) Appl. No.: 18/110,763

(22) Filed: Feb. 16, 2023

(51) Int. Cl. G02C 11/00; G02C 7/04; G02C 7/08

(52) U.S. Cl. CPC G02C 11/10; G02C 7/04; G02C 7/083; G02C 2202/24

(57) ABSTRACT

An ocular system that includes a camera and a display to be inserted intra-ocularly. The camera is preferably located centrally to the display and can include magnification and other functions to assist the user in seeing their environment more clearly. The system can also include pass-through functions such that the user is not disturbed by the presence of the camera in their field of vision.

### FIELD OF THE INVENTION

The field of the invention is intra-ocular camera implants.

### BACKGROUND

The following description includes information that may be useful in understanding the present invention. It is not an admission that any of the information provided herein is prior art or relevant to the presently claimed invention, or that any publication specifically or implicitly referenced is prior art.

Adjustable eye glasses are known that have lenses with fluid filled sac. Typically, diopter is modified using a dial to move fluid between the sacs and reservoirs in the arms of the glasses. Pumping the fluid out of a sac corrects farsightedness, and pumping the fluid into a sac corrects for nearsightedness.

With the rise of increasingly smaller optical equipment and display technology, the possibility of correcting or enhancing a person's vision with intraocular devices has gone from science-fiction to science fact.

However problems in these systems remain. Existing systems do not account for the location of the hardware and how it may affect the user's visibility both when the system is active and also when it is inactive.

Thus, there is still a need for an improved intra-ocular optical system that can enhance the user's vision without being obtrusive or otherwise detrimental to the user when not in use.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The recitation of ranges of values herein is merely intended to serve as a shorthand method of referring individually to each separate value falling within the range. Unless otherwise indicated herein, each individual value is incorporated into the specification as if it were individually recited herein, and ranges include their endpoints.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a.” “an.” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless a contrary meaning is explicitly stated, all ranges are inclusive of their endpoints, and open-ended ranges are to be interpreted as bounded on the open end by commercially feasible embodiments.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### SUMMARY OF THE INVENTION

The inventive subject matter provides apparatus, systems and methods in which an intra-ocular system is implanted in a user's eye. The system includes a display and a camera that can be integrated or otherwise embedded with the display. In most embodiments, the camera is located centrally to the display such that the imagery captured by the camera corresponds to and aligns with the perspective of the user's natural field of vision.

In embodiments, the display is transparent such that the user can see through the display when the display is not displaying imagery. In these embodiments, the display can have a pass-through feature where the display only displays imagery such that the presence of the camera does not disturb a user's field of view.

The display can be used to augment or otherwise enhance the user's vision by changing the brightness, colors, contrast, or other display aspects of the image.

In embodiments, the display can show zoomed-in or otherwise enlarged imagery, such that a user with poor vision can better see the world around them. Similarly, in embodiments, the display can assist users suffering from eye conditions by displaying objects that would otherwise be fuzzy or out-of-focus with clarity.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIGS. 1A, 1B and 1C are diagrammatic front, side and rear views of an optical system according to embodiments of the inventive subject matter.

FIGS. 2A and 2B are diagrammatic front and side views of an optical system according to alternative embodiments of the inventive subject matter.

FIG. 3 shows an illustration of a pass-through mode, according to embodiments of the inventive subject matter.

FIG. 4 shows an illustration of an amplification of a real-world scene as viewed on a display, according to embodiments of the inventive subject matter.

### DETAILED DESCRIPTION

Throughout the following discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions. One should appreciate that the systems and methods of the inventive subject matter offer many technical benefits, including enhancing a user's ability to see their environment without requiring a bulky or otherwise cumbersome external apparatus.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

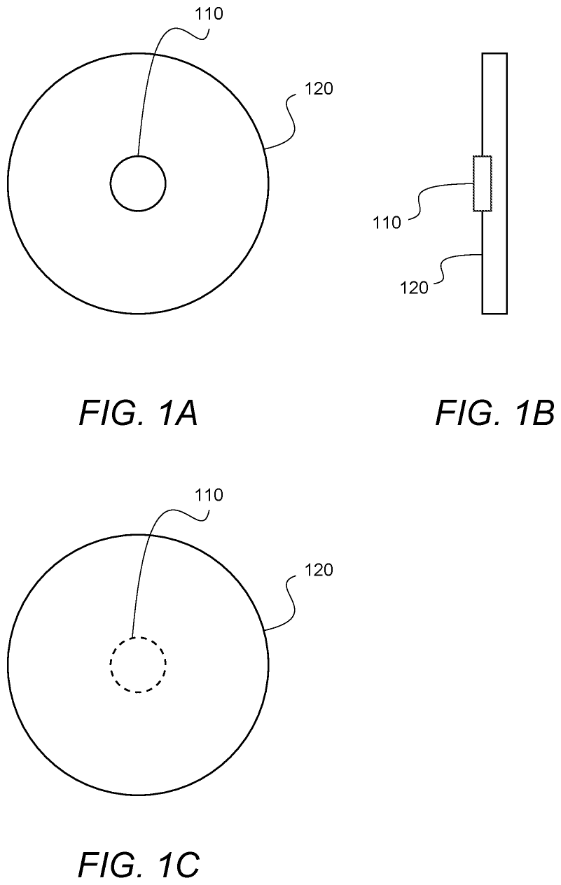

FIGS. 1A-1C generally depict an optical system 100 from a front, side and rear perspective, respectively. The optical system 100 includes a camera 110 on a first side of the optical system 100, and a display screen 120 on a second side of the optical system 100. The system 100 can include one or more processors that can be integrated with the camera 110, the display screen 120 (or both), or another device that can be integral to the system 100 or located outside of the eye that can receive image information and cause the display to display an image visible to the eye of the user. Along with the processors, the system 100 can also include a non-transitory computer-readable memory that can store program instructions and other software-related code that executes the processes discussed herein. The memory can also be used to store imagery (still images and/or video clips) captured by the camera 110.

In FIGS. 1A-1C, the display screen 120 is depicted as being round, which can make for a more natural shape for implanting into the eye. However, it is contemplated that the display 120 can be rectangular in shape as well.

The system 100 is also contemplated to include an eye-mounted power source that provides electrical power to the camera and/or the display.

In embodiments of the inventive subject matter, the system 100 also includes a communication interface that allows the system 100 to wirelessly exchange data with an external device. The wireless communication capabilities can be short range and/or long range, that allow the system 100 to receive updates (such as software updates), receive preferences and user-inputted commands, export imagery and performance reports, etc.

As can be seen in FIGS. 1A and 1B, the camera 110 is disposed on the first side of the optical system 100 in an alignment with the display 120 on the second side of the system 100 such that the image obtained by the camera 110 positionally matches the image as displayed on the display 120. In these embodiments, the system 100 is considered to be centrally disposed within the user's eye. Thus, the arrangement of the camera 110 is such that it is central to the user's eye and their field of vision, and the display 120 is such that to the user the image on the display 120 matches the perspective of what the user's eye would see naturally. In other words, the alignment of the camera 110 and the display 120 is such that the camera captures an image and the display 120 displays the image in a manner that is perceptually concurrent (i.e., to the person wearing the system, it would appear to be simultaneous). For this to occur, the camera is designed to capture video and the display is capable of displaying video at a speed and resolution sufficiently fast that there is no perceptible lag or difference between the real-world outside the eye and the display. It is contemplated that the camera and display are capable of capturing and displaying images, respectively, with a speed of at least 30 frames per second. In other embodiments, the camera and display are capable of capturing and displaying images, respectively, with a speed of at least 60 frames per second In embodiments, this means that the camera 110 will be aligned with the center of the display 120.

In embodiments of the inventive subject matter such as those discussed herein, the display 120 comprises an intraocular lens. In embodiments, the camera can be positioned centrally on the intraocular lens. The display 120 can extend, in embodiments, to one or more of the edges of the intraocular lens such that the display can occupy all or less than all of the field of view offered by the intraocular lens.

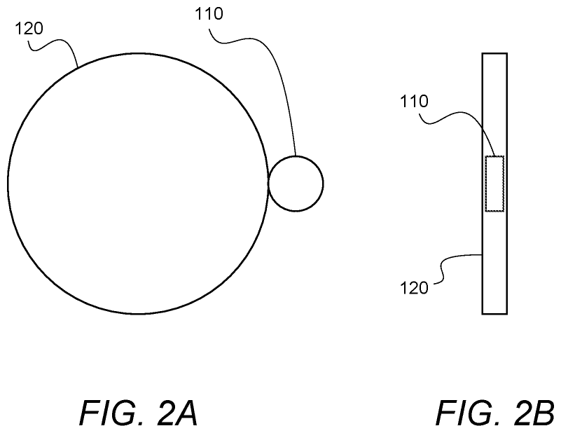

In other embodiments, the camera 110 could be aligned off-center relative to the display 120 if the off-center positioning is small enough to not affect the perspective of the camera relative to the display 120. In these embodiments, the camera 110 could be disposed on a lateral side of the system 100 rather than the front side. In these embodiments, the system 200 can be disposed slightly off-center with the eye such that the camera 110 is centrally aligned with the eye. This way, the image obtained with the camera remains centered from the user's point of view. FIGS. 2A and 2B show front and side views of the system 200 according to these embodiments of the inventive subject matter.

In some of these embodiments, software corrections can be applied to the image from the camera such that it appears to have the same perspective as the naked eye if the system 200 is not centrally disposed in the eye. In a variation of these embodiments, multiple cameras are placed on the lateral sides of the display 120, and their images are aggregated to produce a centralized image.

As seen in the figures, the optical system 100 is thin such that the camera 110 is positioned at least partially within the display 120, such as seen in FIG. 1B. This allows for the camera to have a pass-through feature where the user can see through the camera even when the display is not engaged. The visibility in this mode would be reduced as the camera area is reduced relative to the display screen, but it prevents a total loss of visibility if the display fails or otherwise is powered off.

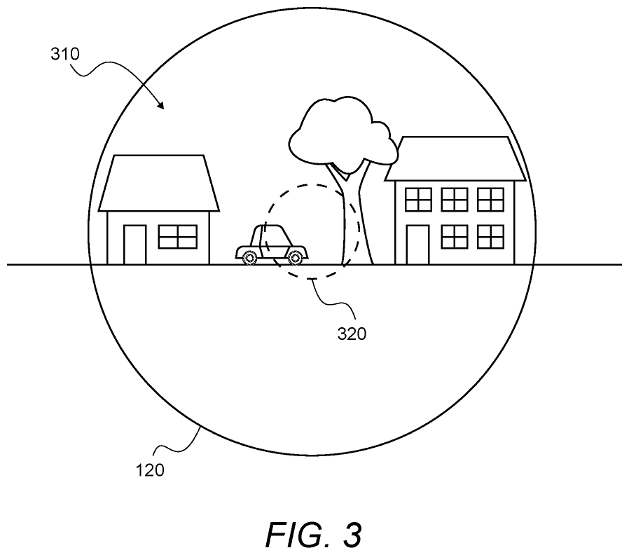

In a variation of these embodiments, the pass-through mode can be activated by the camera 110 and display 120 such that the display 120 only displays imagery directly behind the camera 110. In these embodiments, the display 120 is considered to be a transparent display that would allow a user to see beyond the display 120 when the display 120 is unpowered or otherwise disactivated. The imagery displayed by the display 120 behind the camera 110 corresponds to the part of the field of vision that would be obscured or otherwise obstructed by the placement of the camera 110. The display 120 is programmed to seamlessly integrate this image with the surrounding areas of the display 120 that are not active such the user's vision does not appear to be obstructed. FIG. 3 illustrates this embodiment of the inventive subject matter.

As seen in FIG. 3, there is a visible scene 310 that the user is seeing through the transparent display 120 that is mostly inactive (and thus, completely see-through). The circle 320 shows the area of the display 120 that is active and displaying imagery from the camera 110 such that the camera itself is not visible. The part of the scene 310 visible inside the circle is the imagery captured by the camera 110 and reproduced by the section 320 of the display 120, such that it aligns and matches the rest of the scene 310 from the user's perspective. It should be noted that the circle 320 itself is not visible to the user but only shown for illustrative purposes here.

The display 120 can be any suitable screen that can display an acceptable image at a very small size. Contemplated displays for the display 120 include an LCD or LED screen. In embodiments of the inventive subject matter, the display screen 120 can be curved to better match the natural curvature of the eye.

In embodiments, the display is resiliently flexible. The display 120 can thus be adapted to fit in different parts of the eye and be adapted to fit the curvature accordingly.

In embodiments of the inventive subject matter, the display has an adjustable transparency. The transparency can be adjustable uniformly over the entire display, or can be dynamically adjustable such that the transparency of different sections of the display can be adjusted independently. For example, the display might adjust part of the transparency of the display to be darker or more opaque to account for a glare that is hitting only that portion of the display but leave the rest of the display unadjusted (or adjusted differently) so that the user's vision via the display is otherwise unaffected.

In embodiments of the inventive subject matter, the system 100 is programmed to enhance a user's vision by rendering the images on the display 120 at a greater brightness than the eye would naturally see from the lighting of the environment. This way, a user in a dark environment can have better visibility of the environment than they would with the naked eye. Similarly, users with particularly poor low-light eyesight in places such as certain indoor areas (which would be visible normally to a healthy eye) could have the visibility of lowlight areas enhanced by the increased brightness of the display 120.

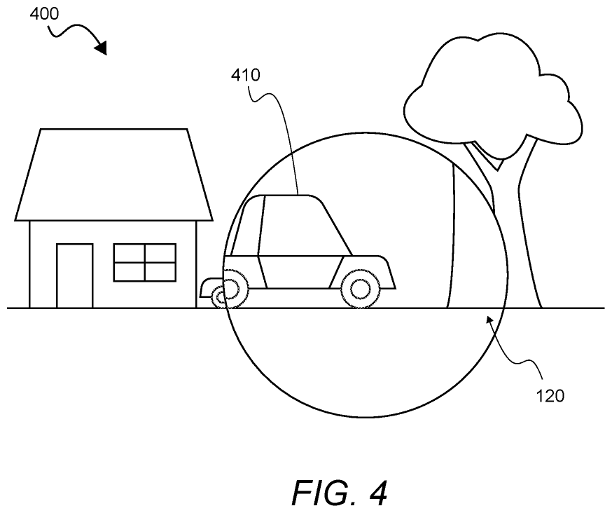

In embodiments of the inventive subject matter, the system 100 is capable of magnifying or amplifying imagery captured by the camera 110 such that the image of the real-world displayed by the display 120 is larger than it would appear naturally via the naked eye. This can be performed via zoom functions of the camera 110 and/or digital zoom techniques executed by the on-board processor. FIG. 4 illustrates this embodiment by showing a field of view 400 that would be visible with the naked eye, with the enlarged sections 410 displayed in the field of view of the display 120. Depending on how much of the user's natural field of view is taken up by display 120, the enlarged sections 410 can take up the user's natural entire field of view or only a portion of it.

In these embodiments, the system 100 can adjust the magnification of the image by the display 120 based on a direction that the user is looking. The direction that the user is looking can be determined by an accelerometer or other position-sensing mechanism that can tell if the user is looking up, down, or turning their heads. For example, if a user is looking at an object that is straight ahead of them, the system 100 can be programmed to render the images from the camera 110 on the display 120 at full magnification. But if a user looks down as determined by the accelerometer, the system 100 is programmed to reduce the magnification so that the user is not disoriented by their legs or feet appearing to be too large or closer than they are in reality. In a variation of these embodiments, the system 100 can determine a distance to an object within the image captured by the camera 110 via image recognition techniques and then amplify the image based on how far the object is.

As discussed herein, the system 100 can be used to aid users who suffer from eye maladies. By having the display 120 render images received by the camera 110, the system 100 can be used to assist users suffering from myopia or presbyopia. For example, the display 120 can make imagery of the real-world environment captured by the camera 110 appear in focus to the user that would appear fuzzy or out of focus via the naked eye. These can be distant objects (for myopic users) or close objects (for presbyopic users). For these users, the imagery can also be manipulated so that objects appear bigger, brighter, with more contrast, darker, more colorful, or otherwise easier for a user to see.

In these embodiments, the system 100 can allow a user to customize how the display 120 renders the image captured by the camera 110 so that the image is displayed according to the user's preferences. For example, via a computer interface that is capable of exchanging data with the system 100, the user can adjust the location of the imagery relative to their eye by moving the displayed images around in the display 120. The user can further adjust interface colors, default brightness and other features.

It is contemplated that, in addition to the processes discussed above, the system 100 can incorporate augmented reality applications. A suitable augmented reality system that can be incorporated here is discussed in applicant's own U.S. patent application Ser. No. 17/103,309 filed Nov. 24, 2020 titled “Augmented Reality Using Intra-Ocular Devices”, which is incorporated herein by reference in its entirety.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An optical system, comprising:
a camera dimensioned to be inserted into a lens of an eye of a human; and
a liquid crystal display dimensioned to be inserted into the lens of the eye, the liquid crystal display juxtaposed with the camera such that an image obtained by the camera positionally aligns with the position of image displayed via the liquid crystal display.

2. The optical system of claim 1, wherein the camera is positioned on a side of the display.

3. The optical system of claim 1, wherein the camera is positioned at least partially within the display.

4. The optical system of claim 1, wherein the display is curved.

5. The optical system of claim 1, wherein the display is resiliently flexible.

6. The optical system of claim 1, wherein the display has adjustable transparency.

7. The optical system of claim 1, wherein the display comprises an intraocular lens.

8. The optical system of claim 7, wherein the camera is positioned central to the intraocular lens.

9. The optical system of claim 7, further comprising an eye-mounted power source that provides electrical power to the camera.

10. The optical system of claim 7, further comprising an eye-mounted power source that provides electrical power to the display.

11. The optical system of claim 7, wherein the display extends to multiple edges of the intraocular lens.

12. A method of providing artificial vision to a user, comprising providing the user with an intra-ocular lens having both a display and a camera.

13. The method of claim 12, further comprising positioning the lens in an eye of the user such that the camera is positioned centrally to a vision path of the eye.

14. The method of claim 12, further comprising powering each of the display and the camera by comprising an eye-mounted power source.

15. The method of claim 12, further comprising improving scotopic vision by using the display to render images received by the camera at a brightness greater than would be received by the eye using a native lens of the eye.

16. The method of claim 12, further comprising using the display to render images received by the camera at a magnification greater than would be received by the eye using a native lens of the eye.

17. The method of claim 12, further comprising at least partially correcting myopia or presbyopia by using the display to render images received by the camera.
